(19) United States
(12) Patent Application Publication
(10) Pub. No.: US 2025/0231134 A1
(43) Pub. Date: Jul. 17, 2025

(54) Indirect, Accurate, and Linear Fluid Level and Moisture Concentration Measurement System and Method Thereof

(71) Applicant: Robert J Bowman, Tijeras, NM (US)

(72) Inventor: Robert J Bowman, Tijeras, NM (US)

(21) Appl. No.: 18/410,411

(22) Filed: Jan. 11, 2024

(51) Int. Cl. G01N 27/22; G01N 33/24; G01R 27/26; H01G 2/22; H01G 4/30

(52) U.S. Cl. CPC G01N 27/223; G01N 27/228; G01N 33/246; G01R 27/2623; H01G 2/22; H01G 4/30

(57) ABSTRACT

A system to provide non-invasive, indirect, continuous, and linear measurement of fluid levels and aggregate soil moisture concentrations. A multilayer device implements a coplanar capacitor capable of measuring fluid levels outside the wall of the vessel containing the fluid. Additionally, the system measures fluid levels and soil moisture concentrations outside the wall of a sealed probe placed in the water or soil. The structure is shielded from surrounding electrical interference, can be manufactured in a cost-effective manner, and be tailored for different physical applications. The invention also includes an electronic circuit for measuring fluid levels and moisture concentrations that represents a robust design in terms of accuracy, noise suppression, calibration, and temperature stability.

### FIELD OF THE INVENTION

The present invention relates generally to a system for the indirect measurement of fluid level and moisture concentration. More specifically, the present invention is a system comprising a coplanar capacitance structure, an electrical circuit, and a plurality of methods to determine fluid level and moisture concentration of vessels and soil, respectively.

### BACKGROUND OF THE INVENTION

Reliable, non-contact, non-invasive, and linear measurement of fluid levels in tanks and moisture concentrations in soil or other material can be achieved using the radiation properties of electric fields. Precision capacitive structures can be designed to sense the presence of fluid levels and soil moisture concentrations through thin wall surfaces without direct contact with the actual fluid or soil moisture. Electronic conditioning circuits convert the variation in capacitance values to simple voltages or digital codes that are linearly related to fluid levels or moisture concentrations.

Prior methods that have been devised to measure fluid levels usually require the sensing devices, such as wire tape, mechanical floats, parallel wires, or piezoelectric pressure sensors, to be in direct contact with the fluid, leading to possible contamination of the fluid or corrosion of the sensor. Non-contact-with-fluid technologies inside the tank such as acoustic wave, ultrasonic, or infrared beam suffer from reliability problems due to transmitters or receivers being contaminated with fluid vapors and condensation.

External tank sensing of fluid levels using a high frequency reflectometer and air dielectric transmission lines require complex electronic conditioning circuits and suffers from external radio and electrical signal interference. Techniques for measuring soil moisture concentration using direct insertion metal soil probes degrade over time due to corrosion. Prior non-contact-soil moisture measurement techniques are limited to surface soil measurement of ten-inch depths or less or by burying small probes at a soil depth requiring long cable runs from the sensor to the data logger.

### SUMMARY OF THE INVENTION

A method in accordance with the embodiments of this invention includes the development of a custom coplanar capacitor structure optimized for non-contact sensing of fluids and moisture. The structure is formed with thin laminations of insulators, adhesives, and metals such that the penetration depth of the electric field pattern is suitable for sensing fluids and moisture through the walls of containment vessels, such as tanks, made of high-density polyethylene, fiberglass, poly vinyl chloride and enclosure walls made with similar vessel wall materials. This method is suitable for both sensing fluid level measurements or moisture concentration of fluid films.

The coplanar capacitor structure in this invention is easily applied to vessel walls or sensor enclosure walls through an adhesive layer. The adhesive layer and insulating layer allow the co-planar capacitor plates to be within 3.3 mils of the contact surface of the vessel wall or sensor enclosure wall. The adhesive layer also seals the front side of the co-planar capacitor. The backside of the capacitive structure is protected and insulated by a 3 mil layer of mylar. This backside surface can also be shielded with a metal layer plus covering insulator to prevent extraneous electrical signals from entering the backside of the coplanar capacitor. The electronic conditioning of the capacitive sensing structure uses reliable, techniques for converting capacitance values into dc voltages or digital codes for data logging of fluid and/or moisture levels.

### BRIEF DESCRIPTION OF THE DRAWINGS

FIG. 1 is a diagram of the components of the present invention.

FIG. 2 is a diagram of the coplanar capacitance structure of the present invention.

FIG. 3 is an alternative diagram of the coplanar capacitance structure, further detailing a length.

FIG. 4 is a diagram of the components of the electronic circuit of the present invention.

FIG. 5 is a wiring diagram of the electronic circuit of the present invention.

FIG. 6 is a diagram of the present invention in a probe configuration, including the housing.

FIG. 7 is a method of the present invention.

FIG. 8 is an alternate method of the present invention.

FIG. 9 is a diagram of an electromagnetic field generated by the present invention.

FIG. 10 is a diagram of the present invention coupled to the exterior wall of a fluid containing vessel.

FIG. 11 is a diagram of the full-scale and zero-scale calibration of the present invention.

FIG. 12 is an additional method of the present invention.

FIG. 13 depicts two tables of performance data for the present invention.

FIG. 14 is an additional method of the present invention.

FIG. 15 is an alternate diagram of the present invention in a probe configuration, including the housing wherein said housing comprises a conical end cap.

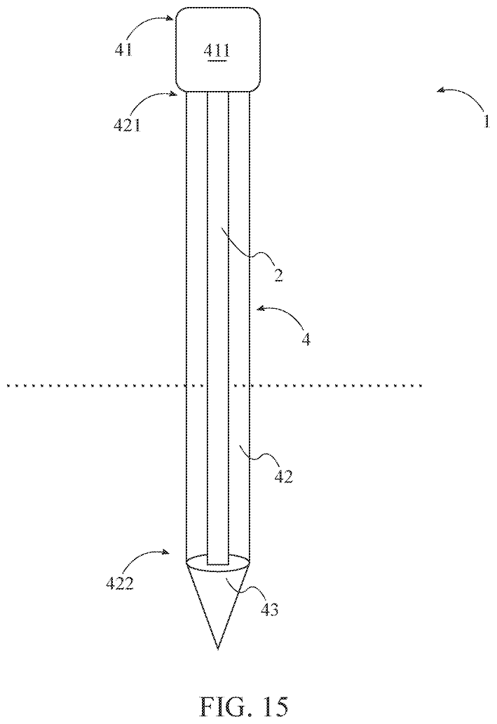

FIG. 16 is a diagram of a plurality of the present invention, measuring soil moisture concentration at various distances away from a stream.

FIG. 17 is a diagram of equations used to tailor the coplanar capacitive structure for different applications.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a system 1 for non-invasive, continuous, and linear measurement of fluid level and moisture concentration, embodiments of the present disclosure are not limited to use only in this context. Explicit design equations are provided to aid in the development of the coplanar capacitor structures for different applications.

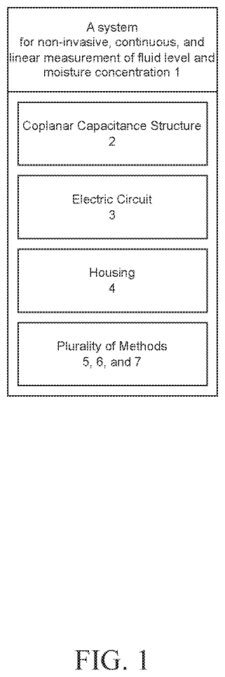

As shown in FIG. 1, the present invention comprises a coplanar capacitance structure 2, an electric circuit 3, and a plurality of methods 5, 6, 7. In some embodiments of the present invention, the system 1 for non-invasive, continuous, and linear measurement of fluid level and moisture concentration further comprises a housing 4.

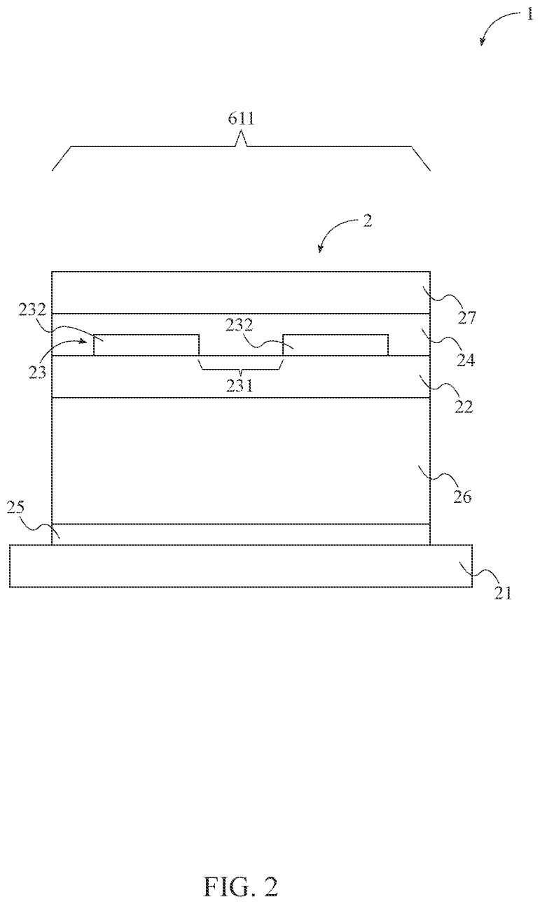

As shown in FIG. 2, the coplanar capacitance structure 2 comprises a tape seal layer 21, an insulated layer 22 comprising an insulating material, wherein said insulating material is mylar, a layer 23 composed of two coplanar strips of conductive material separated by a distance 231, and a layer 24 composed of a polyester film. In the preferred embodiment of the present invention, the strips of conductive material are copper 232. In additional embodiments of the present invention, the coplanar capacitance structure 2, further comprises a metal shield 25, interposed between the tape seal layer 21 and the insulated layer 22. Furthermore, the coplanar capacitance structure 2 may further comprise a thick air cell foam layer 26, wherein said thick air cell foam layer 26 is interposed between the tape seal layer 21 and the insulated layer 22. Lastly, in the preferred embodiment of the present invention, the coplanar capacitance structure 2, comprises an acrylic adhesive layer 27, said acrylic adhesive layer 27 adjacent to the polyester film layer 24.

In the preferred embodiment of the present invention, the tape seal layer 21 is a weatherproof tape. In such preferred embodiment, the tape seal layer 21 is ProTapes Pro Duct 110 PE-Coated Cloth General Purpose Duct Tape or a material of the like.

In some embodiments of the present invention, the air cell foam layer 26 comprises a thickness Tf and a width Wf. Furthermore, in the preferred embodiment of the present invention, the air cell foam layer 26 is Yotache Open Cell Foam Weather-Strip Seal or a material of the like.

In the preferred embodiment of the present invention, the insulated layer 22 is composed of an insulation material wherein said material is a mylar insulation material. In the preferred embodiment of the present invention, the mylar insulation material 22 comprises a width (WF) and a thickness (TF) wherein said thickness is three thousandths of an inch (mils). In some embodiments of the present invention, the insulated layer 22 is greater than three mils dependent upon the application of the system 1. The insulated layer 22 may comprise a mylar thickness of 1-12 mils. Additionally, within some embodiments, the insulated layer 22 may be composed of a foam layer. In the preferred embodiment of the present invention, the mylar insulation material 22 is Dupont Mylar 110 or a material of the like.

Further, within the preferred embodiment of the present invention, the layer of the coplanar capacitance structure 2, composed of the metal shield 25, may comprise a copper plate. In alternate embodiments of the present invention, the metal shield 25 may be composed of a highly conductive and flexible metal such as aluminum, tungsten, or a metal of the like. In the preferred embodiment of the present invention, the metal shield provides a desensitization element to the sensor whereby the metal shield inhibits the sensor from being influenced by extraneous signals. In such embodiments, the metal shield 25 may be adjacent to the air cell foam layer 26.

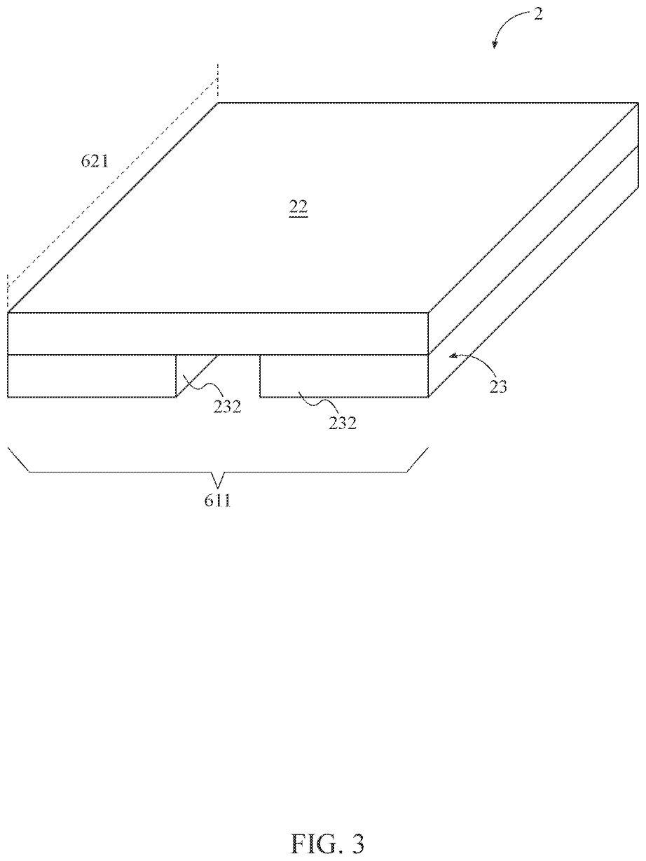

In the context of the present invention, specifically in regard to the layer composed of two coplanar strips 23, the plurality of coplanar conducting material comprises two plates of conducting material 232, a first plate and a second plate, separated by a separation 231, wherein said plates 232 share a common plane 23, as shown in FIG. 2 and FIG. 3. Furthermore, each of the two plates 232, the first plate and the second plate, comprise a width (W1 and W2) wherein each width, in the preferred embodiment is equal (W1=W2=W). The separation 231 between the first plate and the second plate comprises a width wherein said width is 2d. In the preferred embodiment of the present invention, the width of the gap is less than 0.05 inches. Additionally, in the preferred embodiment of the present invention, the width of the plates, W must be at least ten times larger than d. Furthermore, within the context of the present invention, within the preferred embodiment of the present invention, the two plates of conducting material comprises a thickness of 5 to 12 mils.

The polyester film layer 24 of the coplanar capacitance structure 2 comprises a thickness. In the preferred embodiment of the present invention, the thickness of the layer composed of two coplanar strips 23 of the coplanar capacitance structure 2 is two mils. Additionally, in the preferred embodiment of the present invention, the polyester film layer 24 is composed of a polyester material. In some embodiments of the present invention, the polyester film layer 24 covers the two plates 232 of the layer composed of two coplanar strips 23, further comprising lateral sides, whereby the separation 231 is filled by the polyester film layer 24 and the lateral sides of the two plates are also covered, as shown in FIG. 2. Further, within the preferred embodiment of the present invention, the polyester material of the polyester film layer 24 is 8.1 mils in thickness and isolates the two plates of conducting material 232 from the tape seal layer 21.

Additionally, the polyester film layer, in the preferred embodiment of the present invention is a layer of polyester double-sided adhesive tape wherein said tape is TESA 4965 or a material of the like.

In some embodiments of the present invention, the acrylic adhesive layer 27 comprises a thickness of 2.5 mils. Further, the coplanar capacitance structure 2 further comprises a width 611 and a length (L) 621. The length 621 of the coplanar capacitance structure 2 is subject to change given the needs of the application of the present invention. With that, it is necessary for the length of the coplanar capacitance structure 2 to be significantly larger than W and 2d.

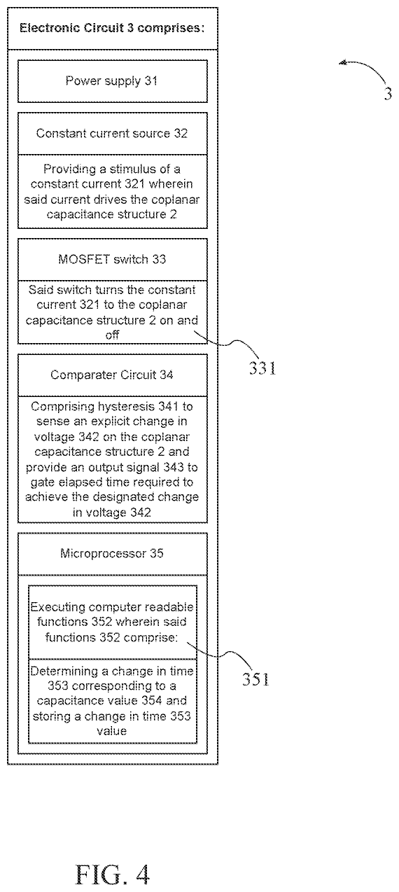

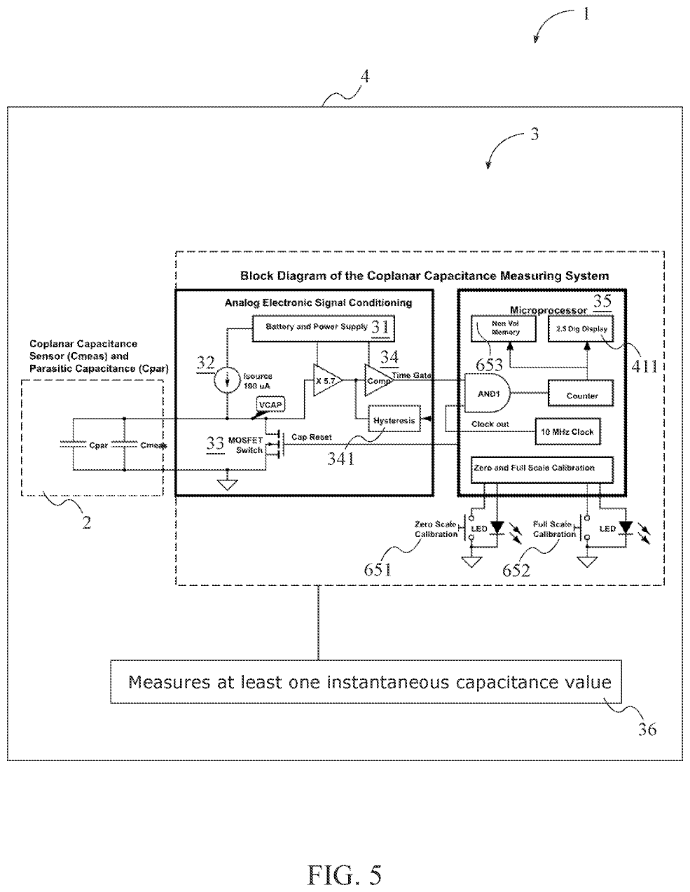

The present invention, as shown in FIG. 4 and FIG. 5, further comprises the electrical circuit 3 wherein said electrical circuit 3 comprises a power supply 31, a constant current source 32, a MOSFET switch 33, a comparator circuit 34, and a microprocessor 35. In the preferred embodiment of the present invention, the constant current source 32 provides a stimulus of a constant current 321 wherein said current drives the coplanar capacitance structure 2. The MOSFET switch 33 turns the constant current 321 to the coplanar capacitance structure 2 on and off. In the preferred embodiment of the present invention the comparator circuit 34 comprises hysteresis 341 to sense an explicit change in voltage 342 on the coplanar capacitance structure 2 and provide an output signal 343 to gate elapsed time required to achieve the designated change in voltage 342. Furthermore, the microprocessor 35 of the electric circuit 3 is a computing device capable of executing 351 computer readable functions and methods 352. In the preferred embodiment of the present invention the computer readable functions and methods 352 comprise determining a change in time 353 corresponding to a capacitance value 354 and storing a change in time value 353. The electrical circuit 3 of the present invention further measures at least one instantaneous value of capacitance 36.

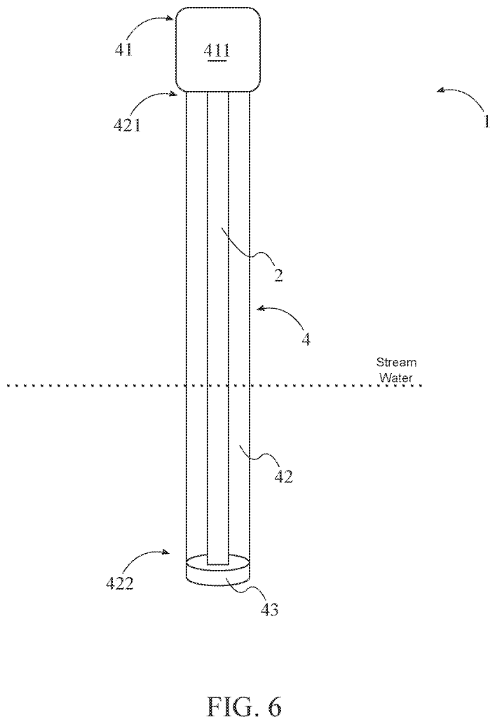

In some embodiments of the present invention, the coplanar capacitance structure 2 and the electrical circuit 3 are housed within the housing 4, as shown in FIG. 5 and FIG. 6. As shown in FIG. 6, the housing 4 of the present invention comprises a head 41, a stem 42, and an end cap 43. The stem 42 comprises two distal ends, a first end 421, and a second end 422, wherein the head 41 is fixed to the first end 421 and the end cap 43 is fixed to the second end 422. In some embodiments, the coplanar capacitance structure 2 and the electrical circuit 3 are housed within the stem 42. In some embodiments, the stem 42 is a PVC tube. In the preferred embodiment, the head 41 comprises a digital display 411 wherein said display 411 is electronically connected to the electrical circuit 3, as shown in FIG. 5. Additionally, in the preferred embodiment, the microprocessor 35 comprises a non-volatile memory unit 653.

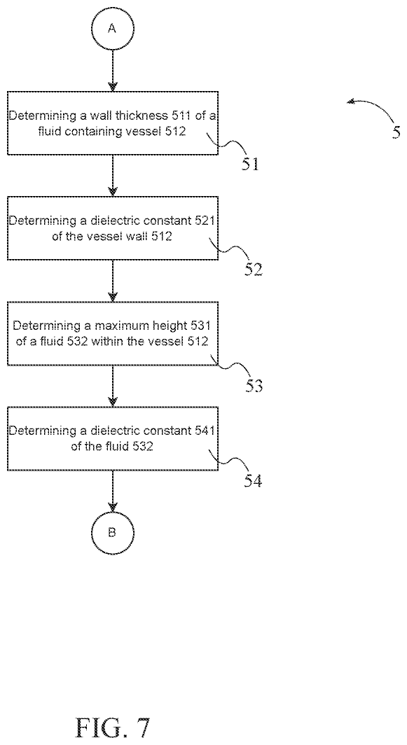

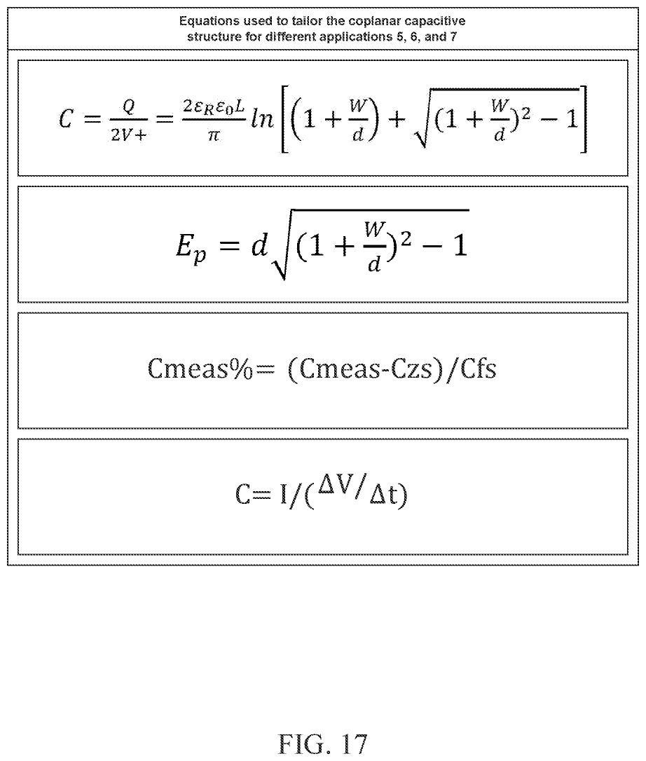

The system 1 of the present invention further comprises the plurality of methods 5, 6, 7 wherein said methods 5, 6, 7 are performed via the microprocessor 35 and the equations as expressed in FIG. 17. A first method 5, as shown in FIG. 7, comprises the steps of: determining 51 a wall thickness 511 of a fluid containing vessel 512;

determining 52 a dielectric constant of the vessel wall 521; determining 53 a maximum height of a fluid 531 within the vessel 512; and determining 54 a dielectric constant of the fluid 541. Because vessels 512 come in various sizes and potable water tanks are often sized to minimize material cost and satisfy the American Society for Testing and Materials (ASTM) standards and specifically, ASTM D 1998 standards. Cylindrical tanks tend to be short in height to minimize static head pressure and a diameter long enough to handle the intended water volume.

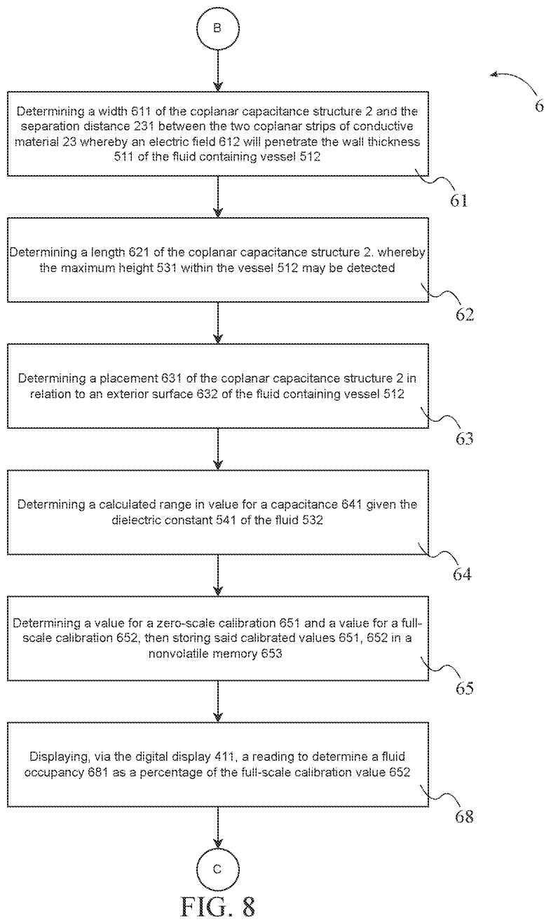

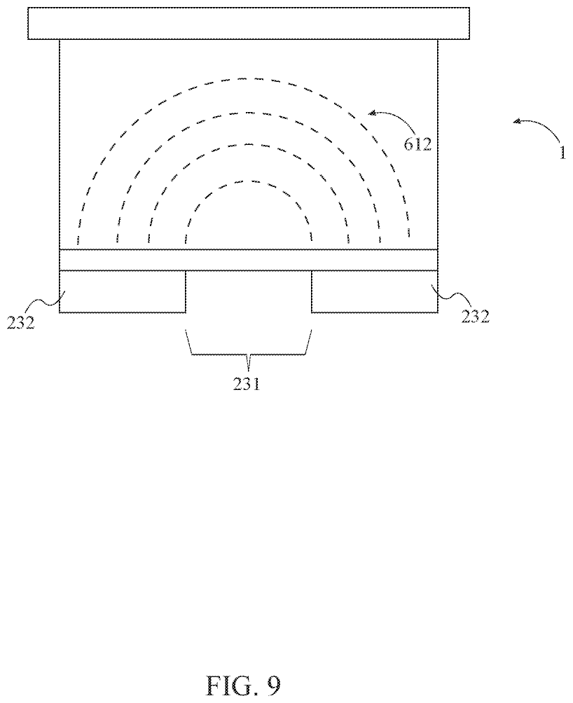

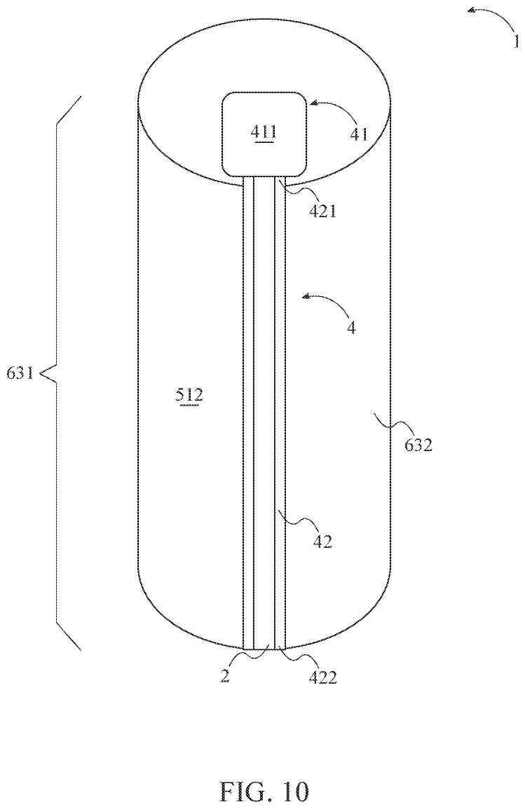

Furthermore, in a second method 6 of the present invention, the system 1, via the microprocessor, performs the steps comprising: determining 61 the width 611 of the coplanar capacitance structure 2 and the separation distance 231 between the two coplanar strips 232 of conductive material whereby an electric field 612 will penetrate the wall thickness 511 of the fluid containing vessel 512, as shown in FIG. 8 and FIG. 9; determining the length 621 of the coplanar capacitance structure 2, whereby the maximum height 531 of the fluid within the vessel 512 may be detected; determining 63 a placement 631 of the coplanar capacitance structure 2 in relation to an exterior surface 632 of the fluid containing vessel 512, as shown in FIG. 10; determining 64 a calculated range in value for a capacitance 641 given the dielectric constant of the fluid 541; and determining 65 a value for a zero-scale calibration 651 and a value for a full-scale calibration 652, and storing 653 said calibrated values in the nonvolatile memory; and displaying 68, via the digital display 411, a reading to determine a fluid occupancy as a percentage of the full-scale calibration value 681. As shown in FIG. 9, the electric field contours 612 extend above the plates 232 and may be referred to herein as fringing capacitance. In describing the capacitance behavior, a simplified analysis leads to information regarding the penetration depth of the fringing field Ep, width of the parallel plates W, and significance of fluid film thickness, Ft. Using conformal mapping techniques known to those in the art, the two-dimensional electric field distribution can be described, as shown in the equations expressed in FIG. 17. The results for semi-infinite electrodes or where L>>W, and with small separation defined as W/d>>1, leads to the equation expressed in FIG. 17. The aforementioned equation provides a reliable estimate of the capacitance for a coplanar electrode pair of finite width provided W/d>>1. The capacitance increases as the natural log of the dimensions instead of linearly for the parallel plate structure so the challenge is to widen the width of plates and keep the separation distance small to achieve the desired electric field penetration Ep for a non-contact (indirect) sensor. The field penetration depth can be deduced from the elliptical contours. The penetration depth, Ep, corresponds to the maximum vertical displacement of the field line 612 emanating from the outermost edge of the coplanar strips 232 as shown in FIG. 9 and described in the equations expressed in FIG. 17 assuming fluid film thickness Ft>Ep.

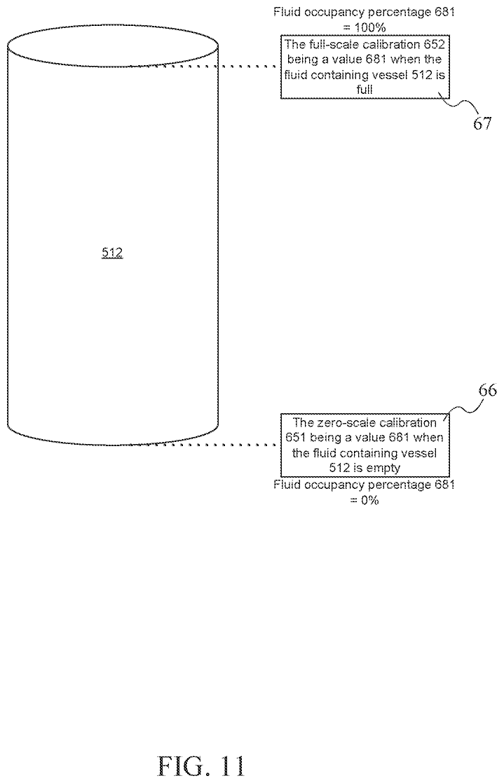

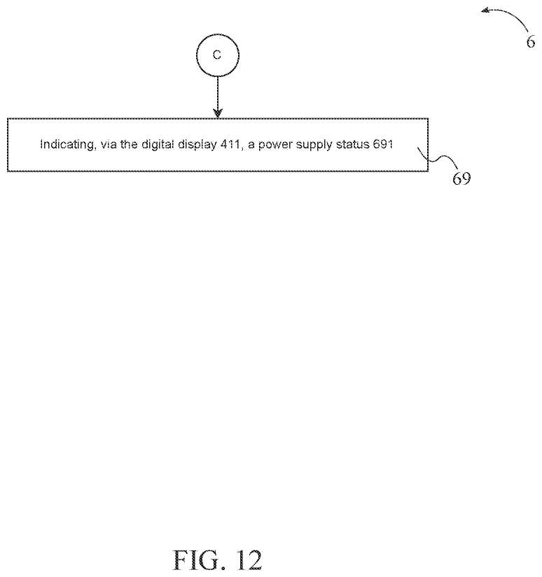

As shown in FIG. 11, the zero-scale calibration 651 value being a value of capacitance when the fluid containing vessel 512 is empty 66; and the full-scale calibration value 652 being a value of capacitance when the fluid containing vessel 512 is full 67. In some embodiments of the present invention, the second method 6 of the system 1 further comprises a step of indicating 69 a power supply 31 status, via the digital display 411, as shown in FIG. 12. Additionally, in the embodiments of the present invention, wherein the system 1 is used to determine fluid occupancy of a fluid container vessel 512, it is preferred that the coplanar capacitance structure 2 is attached to the outer wall 632 of the vessel 512.

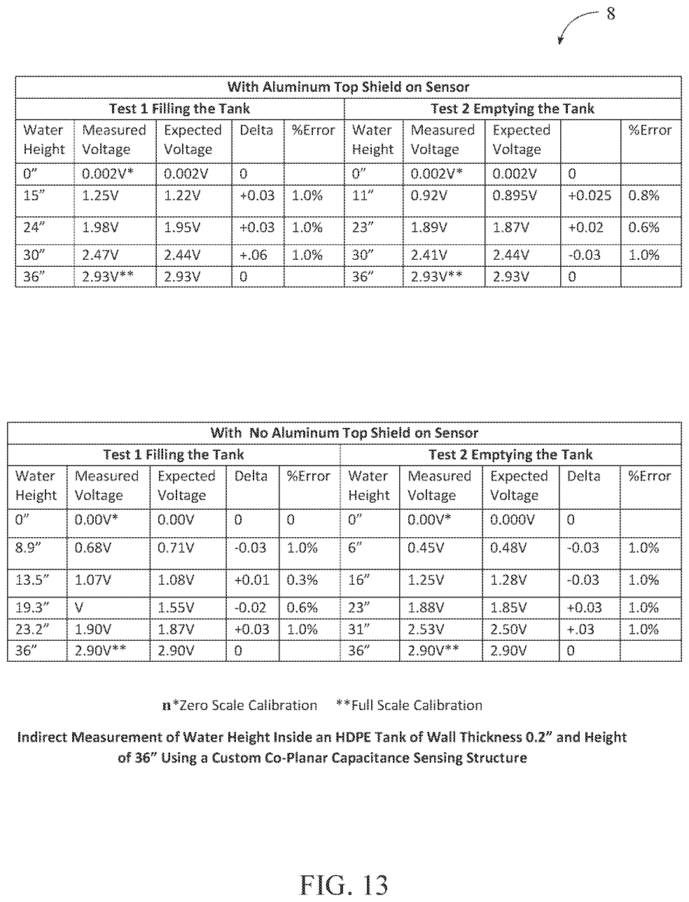

As shown by a plurality of tables 8, as in FIG. 13, the performance of the present invention for measuring the water height inside an HDPE container of height 36″ and wall thickness 0.2″, the results indicate a measurement accuracy and linearity within +/−1%.

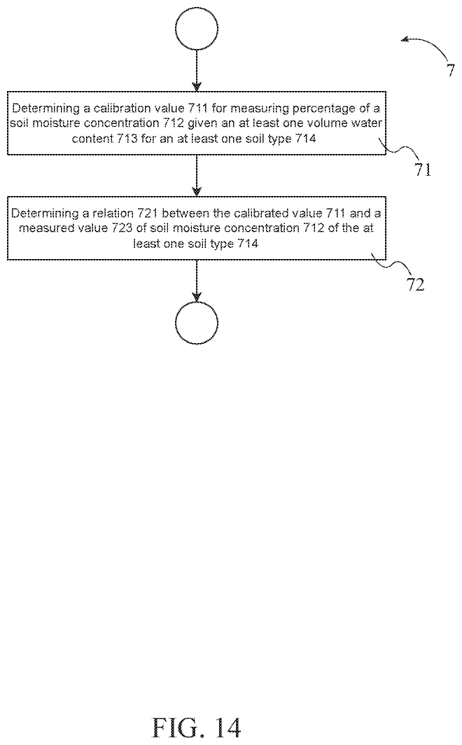

In a third method 7 of the present invention, the system 1 further carries out the steps including: determining 71 a calibration value 711 for measuring percentage of a soil moisture concentration 712 given at least one volume water content 713 for at least one soil type 714; and determining 72 a relation between the calibration value 711 and a measured value of soil moisture concentration 723 of the at least one soil type 714, as shown in FIG. 14.

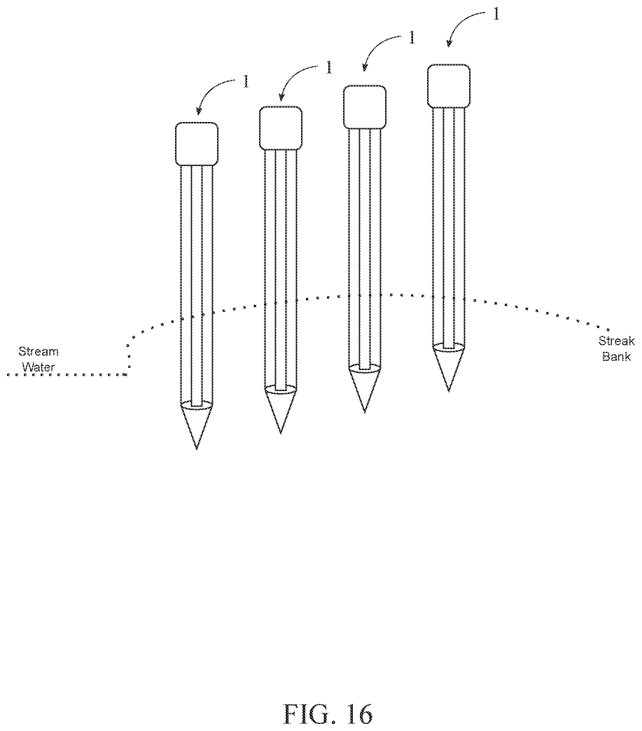

In embodiments of the present invention, wherein the system 1 is used to measure soil moisture concentration 7, as shown in FIG. 14, the present invention may further include an endcap 43 wherein said end cap 43 comprises a conical shape. As shown in FIG. 16, in some embodiments of the present invention, a plurality, or an array, of the present invention 1, may be used to detect the presence of water. In said embodiments, as the water rises or falls in the stream, the coplanar capacitor changes in capacitance proportional to the stream height by sensing the change in dielectric from air to water.

This is accomplished indirectly through the wall of the stem 42. Each stream height reading can be date and time logged for future data acquisition. The array of the present invention 1, as shown in FIG. 16, along a stream bank may indirectly make measurement of soil moisture 723, thus protecting the watershed from contamination. Understanding the distribution of moisture content along stream banks is very important in assessing the techniques used to restore riparian habitats after streams have been damaged by forest fires, floods, or droughts. The present invention may be placed at uniform distances from the stream edge with increasing depths to measure the permeation of water into the bank after stream restoration methods have been applied. These stream restoration methods may include physical stream path modifications, artificial beaver dams, and small rock dams, all intended to slow the stream flow of water to increase the watershed area.

Although the invention has been explained in relation to its preferred embodiment, it is to be understood that many other possible modifications and variations can be made without departing from the spirit and scope of the invention.

What is claimed is:

1. A system for non-invasive, continuous, and linear measurement of fluid level and moisture concentration comprising a coplanar capacitance structure wherein said structure comprises:
a tape seal layer composing a bottom layer;
an insulated layer comprising an insulating material, wherein said insulating material is mylar;
a layer composed of two coplanar strips of conductive material, separated by a distance; and
a layer composed of a polyester film, composing a topmost layer;
the insulated layer being adjacent to the tape seal layer;
the layer composed of two coplanar strips being adjacent to the insulated layer;
the polyester film layer being adjacent to the layer composed of two coplanar strips;
the tape seal layer and the polyester film layer composing the two outermost layers, wherein the insulated layer and layer composed of two coplanar strips are interposed in between said outermost layers.

2. The system for non-invasive, continuous, and linear measurement of fluid level and moisture concentration, as claimed in claim 1, wherein the coplanar capacitance structure further comprises a metal shield, interposed between the tape seal layer and the insulated layer.

3. The system for non-invasive, continuous, and linear measurement of fluid level and moisture concentration, as claimed in claim 1, wherein the coplanar capacitance structure further comprises a thick air cell foam layer, wherein said thick air cell foam layer is interposed between the tape seal layer and the insulated layer.

4. The system for non-invasive, continuous, and linear measurement of fluid level and moisture concentration, as claimed in claim 1, wherein the coplanar capacitance structure further comprises an acrylic adhesive layer, said acrylic adhesive layer adjacent to the polyester film layer.

5. The system for non-invasive, continuous, and linear measurement of fluid level and moisture concentration, as claimed in claim 1, wherein the two coplanar strips of conductive material are copper.

6. The system for non-invasive, continuous, and linear measurement of fluid level and moisture concentration as claimed in claim 1, further comprising an electric circuit, wherein said electronic circuit comprises:
a power supply;
a constant current source;
a MOSFET switch;
a comparator circuit;
a microprocessor;
the coplanar capacitance structure serves as an input device to said electric circuit;
said electric circuit measures at least one instantaneous value of capacitance;
the constant current source providing a stimulus of a constant current wherein said current drives the coplanar capacitance structure;
the MOSFET switch turning the constant current to the coplanar capacitance structure on and off;
the comparator circuit comprising hysteresis to sense an explicit change in voltage on the coplanar capacitance structure and provide an output signal to gate elapsed time required to achieve the designated change in voltage; and
the microprocessor capable of executing computer readable functions and methods wherein said functions and methods comprise:
determining a change in time corresponding to a capacitance value and storing a change in time value.

7. The system for non-invasive, continuous, and linear measurement of fluid level and moisture concentration as claimed in claim 6, wherein the coplanar capacitance structure and the electronic circuit are contained within a housing, wherein said housing comprises:
a head;
a stem; and
an end cap;
the stem being a tube comprising two opposite distal ends, a first end and a second end, wherein the head is fixed to the first end and the end cap is fixed to the second end;
the head comprising a digital display;
the coplanar capacitance structure and the electronic circuit being housed within the stem; and
the digital display being electronically connected to the electronic circuit.

8. The system for non-invasive, continuous, and linear measurement of fluid level and moisture concentration, as claimed in claim 6, comprising the steps of:
determining a wall thickness of a fluid containing vessel;
determining a dielectric constant of the vessel wall;
determining a maximum height of a fluid within the vessel; and
determining a dielectric constant of the fluid.

9. The system for non-invasive, continuous, and linear measurement of fluid level and moisture concentration, as claimed in claim 8, comprising the steps of:
determining a width of the coplanar capacitance structure and the separation distance between the two coplanar strips of conductive material whereby an electric field will penetrate the wall thickness of the fluid containing vessel;
determining a length of the coplanar capacitance structure, whereby the maximum height of the fluid within the vessel may be detected;
determining a placement of the coplanar capacitance structure in relation to an exterior surface of the fluid containing vessel;
determining a calculated range in value for a capacitance given the dielectric constant of the fluid; and
determining a value for a zero-scale calibration and a value for a full-scale calibration, and storing said calibrated values in a nonvolatile memory;
the zero-scale calibration value being a value of capacitance when the fluid containing vessel is empty; and
the full-scale calibration value being a value of capacitance when the fluid containing vessel is full;
displaying, via the digital display, a reading to determine a fluid occupancy as a percentage of the full-scale calibration value; and
indicating, via the digital display, a power supply status.

10. The system for non-invasive, continuous, and linear measurement of fluid level and moisture concentration as claimed in claim 7, comprising the steps:
determining a calibration value for measuring percentage of a soil moisture concentration given at least one volume water content for at least one soil type; and
determining a relation between the calibration value and a measured value of soil moisture concentration of the at least one soil type.

11. A system for non-invasive, continuous, and linear measurement of fluid level and moisture concentration comprising a coplanar capacitance structure wherein said structure comprises:
a tape seal layer composing a bottom layer;
an insulated layer comprising an insulating material, wherein said insulating material is mylar;
a layer composed of two coplanar strips of conductive material, separated by a distance;
a layer composed of a polyester film, composing a topmost layer; and
an electronic circuit;
the insulated layer being adjacent to the tape seal layer;
the layer composed of two coplanar strips being adjacent to the insulated layer;
the polyester film layer being adjacent to the layer composed of two coplanar strips;
the tape seal layer and the polyester film layer composing the two outermost layers, wherein the insulated layer and the layer composed of two coplanar strips are interposed in between said outermost layers;
the coplanar capacitance structure serving as an input device to said electric circuit; and
said electric circuit measures at least one instantaneous value of capacitance.

12. The system for non-invasive, continuous, and linear measurement of fluid level and moisture concentration as claimed in claim 11, wherein the coplanar capacitance structure and the electronic circuit are contained within a housing, wherein said housing comprises:
a head;
a stem; and
an end cap;
the stem being a tube comprising two opposite distal ends, a first end and a second end, wherein the head is fixed to the first end and the end cap is fixed to the second end;
the head comprising a digital display;
the coplanar capacitance structure and the electronic circuit being housed within the stem; and
the digital display being electronically connected to the electronic circuit.

13. The system for non-invasive, continuous, and linear measurement of fluid level and moisture concentration, as claimed in claim 11, wherein the coplanar capacitance structure further comprises a metal shield, interposed between the tape seal layer and the insulated layer.

14. The system for non-invasive, continuous, and linear measurement of fluid level and moisture concentration, as claimed in claim 11, wherein the coplanar capacitance structure further comprises a thick air cell foam layer, wherein said thick air cell foam layer is interposed between the tape seal layer and the insulated layer.

15. The system for non-invasive, continuous, and linear measurement of fluid level and moisture concentration, as claimed in claim 11, wherein the coplanar capacitance structure further comprises an acrylic adhesive layer, said acrylic adhesive layer adjacent to the polyester film layer.

16. The system for non-invasive, continuous, and linear measurement of fluid level and moisture concentration, as claimed in claim 11, comprising the steps of:
determining a wall thickness of a fluid containing vessel;
determining a dielectric constant of the vessel wall;
determining a maximum height of a fluid within the vessel; and
determining a dielectric constant of the fluid.

17. The system for non-invasive, continuous, and linear measurement of fluid level and moisture concentration, as claimed in claim 16, comprising the steps of:
determining a width of the coplanar capacitance structure and the separation distance between the two coplanar strips of conductive material whereby an electric field will penetrate the wall thickness of the fluid containing vessel;
determining a length of the coplanar capacitance structure, whereby the maximum height of the fluid within the vessel may be detected;
determining a placement of the coplanar capacitance structure in relation to an exterior surface of the fluid containing vessel;
determining a calculated range in value for a capacitance given the dielectric constant of the fluid; and
determining a value for a zero-scale calibration and a value for a full-scale calibration, and storing said calibrated values in a nonvolatile memory;
the zero-scale calibration value being a value of capacitance when the fluid containing vessel is empty; and
the full-scale calibration value being a value of capacitance when the fluid containing vessel is full.

18. The system for non-invasive, continuous, and linear measurement of fluid level and moisture concentration as claimed in claim 12, comprising the steps:
determining a calibration value for measuring percentage of a soil moisture concentration given at least one volume water content for at least one soil type; and
determining a relation between the calibration value and a measured value of soil moisture concentration of the at least one soil type.

19. A system for non-invasive, continuous, and linear measurement of fluid level and moisture concentration comprising a coplanar capacitance structure wherein said structure comprises:
a tape seal layer composing a bottom layer;
an insulated layer comprising an insulating material, wherein said insulating material is mylar;
a layer composed of two coplanar strips of conductive material, separated by a distance;
a layer composed of a polyester film, composing a topmost layer; and
an electronic circuit;
the insulated layer being adjacent to the tape seal layer;
the layer composed of two coplanar strips being adjacent to the insulated layer;
the polyester film layer being adjacent to the third layer;
the tape seal layer and the polyester film layer composing the two outermost layers, wherein the insulated layer and the layer composed of two coplanar strips are interposed in between said outermost layers;
the coplanar capacitance structure serving as an input device to said electric circuit; and
said electric circuit measures at least one instantaneous value of capacitance; and
the electronic circuit and the coplanar capacitance structure further:
determining a width of the coplanar capacitance structure and the separation distance between the two coplanar strips of conductive material whereby an electric field will penetrate a wall thickness of a fluid containing vessel;
determining a length of the coplanar capacitance structure,
whereby a maximum height of a fluid within the vessel may be detected;
determining a placement of the coplanar capacitance structure in relation to an exterior surface of the fluid containing vessel;
determining a calculated range in value for a capacitance given a dielectric constant of the fluid; and
determining a value for a zero-scale calibration and a value for a full-scale calibration, and storing said calibrated values in a nonvolatile memory;
the zero-scale calibration value being a value of capacitance when the fluid containing vessel is empty; and
the full-scale calibration value being a value of capacitance when the fluid containing vessel is full.

20. The system for non-invasive, continuous, and linear measurement of fluid level and moisture concentration, as claimed in claim 19, comprising the steps of:
determining the wall thickness of the fluid containing vessel;
determining the dielectric constant of the vessel wall;
determining the maximum height of the fluid within the vessel; and
determining the dielectric constant of the fluid.
